(19) United States
(12) Patent Application Publication
(10) Pub. No.: US 2023/0018733 A1
(43) Pub. Date: Jan. 19, 2023

(54) BRACE

(71) Applicant: ALLEVIATE, INC., Boston, MA (US)

(72) Inventors: Sean M. McDowell, Winchester, MA (US); Roderick McMullen, Boston, MA (US); Luke Ferdinands, Newton, MA (US)

(21) Appl. No.: 17/944,767

(22) Filed: Sep. 14, 2022

Related U.S. Application Data
(63) Continuation of application No. PCT/US2021/033634, filed on May 21, 2021.
(60) Provisional application No. 63/028,217, filed on May 21, 2020.

(51) Int. Cl. A61F 5/01

(52) U.S. Cl. CPC A61F 5/0111; A61F 5/0127; D10B 2403/023

(57) ABSTRACT

A brace may include a knit portion with an ankle strap, a rigid shank on a bottom portion of the brace configured to cover at least an arch portion of the foot, a pad positioned below a heel of the foot, a webbing support system extending from the lateral extensor digitorum longus tendon around the peroneus longus retinaculum tendon, under the bottom of the foot to support the plantar tendon and up the arch and medial side of the foot to the extensor hallucis longus tendon, a lacing system threaded through a portion of the webbing support system at the top medial portion of the foot, and a tightening system that applies a pulling force on the lacing system to cinch the webbing support system.

### CROSS-REFERENCE TO RELATED APPLICATIONS

This application is a bypass continuation of International Application Serial No. PCT/US2021/033634 filed May 21, 2021 and titled “BRACE” (ALLV-0001-WO).

International Application Serial No. PCT/US2021/033634 claims priority to U.S. Provisional Patent Application Ser. No. 63/028,217, filed May 21, 2020 and titled “ANKLE BRACE” (ALLV-0001-P01).

All of the above patent documents are incorporated herein by reference in their entirety.

### BACKGROUND

### Field

The current disclosure relates to providing a custom or pre-fabricated brace. The brace provides tri-planar control of ankle and foot structures inducing stability and control to the ankle, hind-foot, mid-foot, and bottom of the foot. The current disclosure also relates to systems, apparatus, and methods that mitigate plantar fasciitis, Achilles tendonitis, pronation of ankle, medial arch collapse, valgus deformity of the knee, posterior tibial tendon dysfunction (PTTD), foot arthritis, ankle weakness or instability of the ankle-foot structure.

### Description of the Related Art

Typical braces use bulky and cumbersome straps to hold the ankle and foot in place, which prevents natural motion, can cause irritation to the skin and may reduce mobility. Another solution for orthotic support is a custom made orthotic from a podiatrist, however, these can be expensive and time consuming to produce. Finally, there are several thin sleeve solutions that are ineffective in generating stability of the foot or ankle.

There remains a need for a brace that replicates a custom taping system effective at reducing foot and ankle dysfunction while allowing for foot and ankle mobility and stability.

### SUMMARY

Embodiments of the current disclosure provide for a brace, also referred to herein as an ankle brace, that includes a knit portion, a shank, and a support system. The knit portion is contoured for a foot. The shank is contoured for a bottom of the foot so as to cover at least a portion of an arch of the foot. The support system extends along at least part of the knit portion and is structured to generate a force between the shank and the foot.

In embodiments, the knit portion, much like a sock, is contoured to correspond to an ankle and foot structure and may have openings for breathability and comfort. Joined to the knit portion is an ankle strap to secure the brace to a user's ankle. The ankle strap may feature any number of closing mechanisms, such as snaps, laces, or hook and loop fasteners, and the ankle strap may be made from at least one of a leather, a synthetic leather, a suede, a synthetic suede, a microfiber, or a combination thereof. The knit portion may include a finishing on an edge of the knit portion, wherein the finishing includes one or more of a binding tape, a flat knit stitch or a welded edge.

In embodiments, a rigid shank may be adhered to the knit portion or to a portion of the knit structure positioned on the bottom of a foot. The rigid shank may be sized to extend from the heel to cover at least an arch portion of the foot and may be a woven structure, such as a cradle weave, and may have a horseshoe shape around the heel. The rigid shank is relatively thin, ranging between about 1 mm to about 2.5 mm, with the possibility of being thicker than 2.5 mm. The rigid shank may be flat on the lateral side of the foot to decrease irritation, but may wrap the foot medially to support the arch. The rigid shank may include one or more of a glass-reinforced nylon, a polyether block amide (PEBA) or other thermoelastic polymer, a thermoplastic elastomer, a thermoplastic urethane, an ethylene vinyl acetate, a poly (ethylene vinyl acetate), a carbon fiber or a rigid plastic, and may include a plurality of openings to allow for comfort and breathability.

A pad, made from a soft, closed cellular foam urethane is positioned below a heel of the foot and adhered to at least one of the knit portion or the rigid shank. The pad can be tear-drop shaped so that it provides comfort and cushioning directly under the center of the heel, and as the shape tapers, it creates a transition, providing a cushioning and representation/functioning of the anatomical structures of the musculotendinous junction of the plantar fascia.

In embodiments, the brace features a webbing support system, which may be a single continuous piece of webbing, ribbon or cord, or may be a plurality of portions of webbing, wherein portions of the webbing support system may extend from the lateral extensor digitorum longus tendon around the peroneus longus retinaculum tendon, under the bottom of the foot to support the plantar tendon and up the arch and medial side of the foot to the extensor hallucis longus tendon. This creates a cradle support system that mimics a physical therapist's taping structure. The webbing support system may include a nylon band, an elastomeric band, a polyester yarn, a ribbon, a cord, or a thermoplastic band. The webbing support system may weave through one or more openings in the knit portion, and may pass between the rigid shank and the knit portion. The webbing support system may form a plurality of loops at the top medial portion of the foot, or if formed of individual webbing portions, the individual webbing portions may each terminate in a portion configured to be threaded by a lacing system. A TPU film may be welded over the plurality of loops to secure them in place and add support to the medial and lateral sides of the brace.

In embodiments, a lacing system may be threaded through the portion of the webbing support system at the top medial portion of the foot, such as the loops formed by the webbing support system or an eyelet or other attachment point on the webbing support system portions. The lacing system includes a lace or a plurality of laces comprising one or more of a cotton, a polyester or a nylon cord. Portions of the lacing system attach to or terminate in a tightening system.

In embodiments, the tightening system applies a pulling force on the lacing system to cinch the webbing support system. The tightening system may include a ratchet system, a barrel lock or an adjustable clip. The tightening system may be located on the ankle strap of the brace or the knit portion. Individual laces of the lacing system may attach to one of a plurality of tightening systems for adjustment of the portion of the webbing support system threaded through or otherwise associated with the individual laces.

In embodiments, the brace may also include at least one protrusion disposed anywhere on the inside of the knit portion to help reduce slipping and to ensure the brace stays in the correct location during activity. The protrusion may include a silicon bead.

Other embodiments of the current disclosure may provide for a method of manufacturing a brace. The method includes securing a knit portion to a shank. The knit portion may be contoured for a foot and the shank may be contoured for a bottom of the foot so as to cover at least a portion of an arch of the foot. A support system is secured to at least one of the knit portion or the shank.

Yet other embodiments of the current disclosure provide for a method of bracing a foot. The method includes inserting the foot into a knit portion of a brace such that at least a portion of an arch of the foot is covered by a shank of the brace; and generating, via a support system of the brace, a force between the shank and the foot. The support system may extend along at least a part of the knit portion.

These and other systems, methods, objects, features, and advantages of the present disclosure will be apparent to those skilled in the art from the following detailed description of the preferred embodiment and the drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE FIGURES

The disclosure and the following detailed description of certain embodiments thereof may be understood by reference to the following figures:

FIG. 1 is a perspective view of a brace, in accordance with an embodiment of the current disclosure;

FIG. 2 is a bottom view of the brace of FIG. 1, in accordance with an embodiment of the current disclosure;

FIG. 3 is a perspective view of the bottom of the brace of FIG. 1, in accordance with an embodiment of the current disclosure;

FIG. 4 is a lateral view of the brace of FIG. 1, in accordance with an embodiment of the current disclosure;

FIG. 5 is a top-down view of the brace of FIG. 1, in accordance with an embodiment of the current disclosure;

FIG. 6 is a back view of the brace of FIG. 1, in accordance with an embodiment of the current disclosure;

FIG. 7 is a perspective view of another embodiment of the brace of FIG. 1, in accordance with the current disclosure;

FIG. 8 is a lateral view another embodiment of the brace of FIG. 1, in accordance with the current disclosure;

FIG. 9 is a front view of the brace of FIG. 8, in accordance with an embodiment of the current disclosure;

FIG. 10 is a bottom view of the brace of FIG. 8, in accordance with an embodiment of the current disclosure;

FIG. 11 is a perspective view of the bottom of the brace of FIG. 8, in accordance with an embodiment of the current disclosure;

FIG. 12 is a flow chart depicting a method for manufacturing a brace, in accordance with an embodiment of the current disclosure;

FIG. 13 is another flow chart depicting the method of FIG. 12, in accordance with an embodiment of the current disclosure; and

FIG. 14 is a flow chart depicting a method of bracing a foot, in accordance with an embodiment of the current disclosure.

### DETAILED DESCRIPTION

In an aspect, certain ankle or foot pathologies, such as plantar fasciitis or tendonitis, posterior tibial tendon dysfunction (PTTD), foot arthritis, ankle weakness or instability of the ankle-foot structure, may be mitigated by various taping methods (e.g. kinesiology taping, rehabilitative taping, therapeutic taping, etc.) and other rehabilitative or therapeutic approaches. Braces of the current disclosure may be effective at replicating a custom taping solution to provide stability and control to ankle foot structures. The brace may be light, breathable, strong and supportive. In embodiments, the brace may reduce weight displacement on the plantar tendon and mitigate and encourage recovery from various foot and ankle dysfunctions.

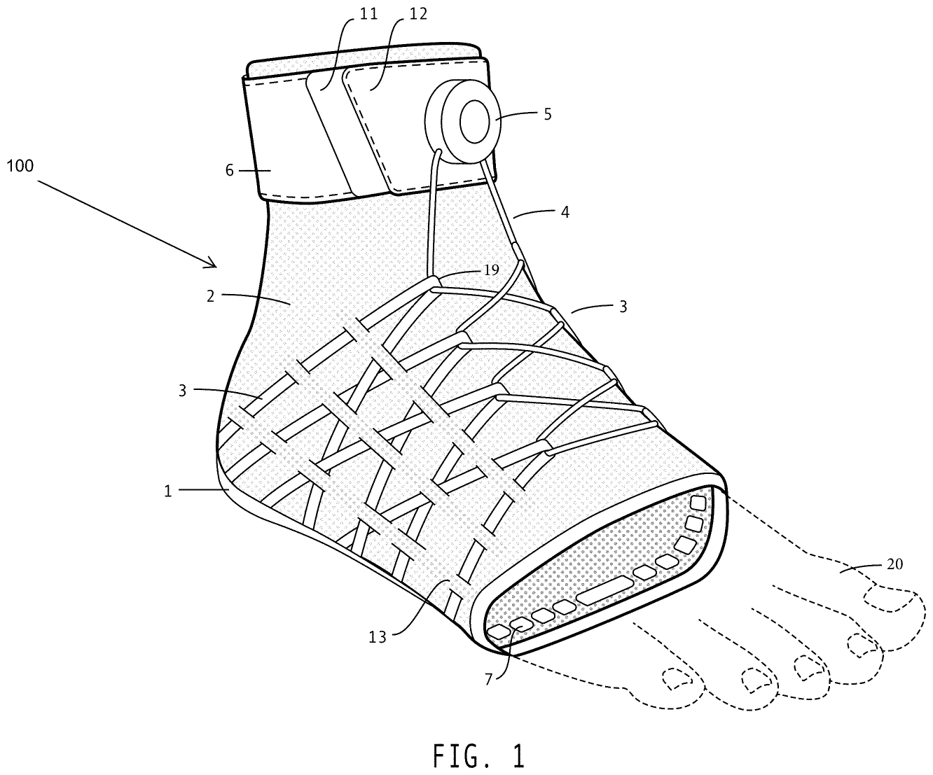

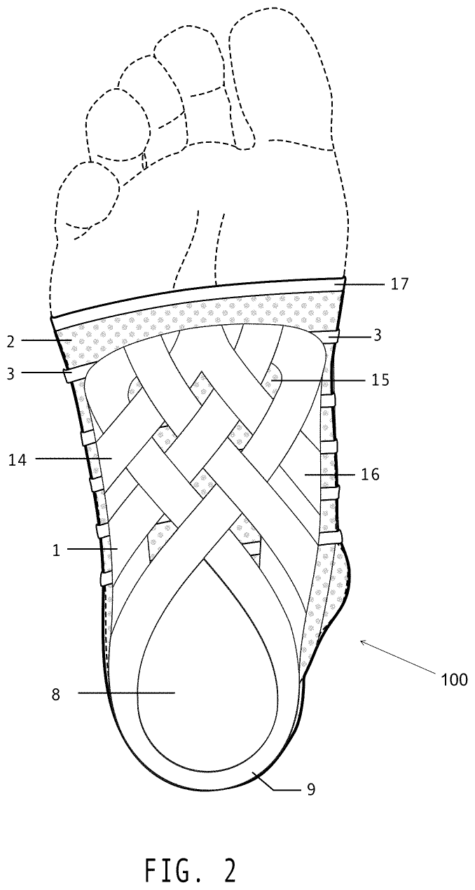

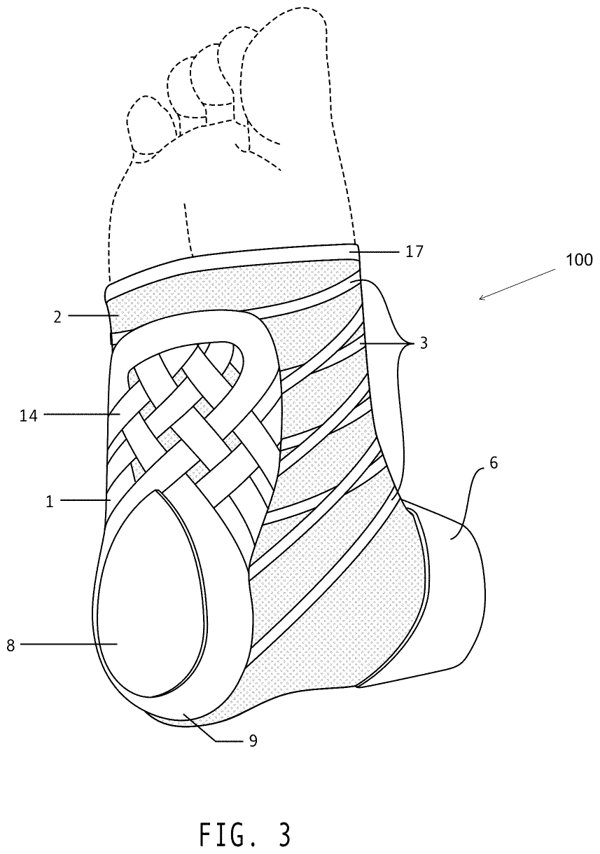

In embodiments, and referring to FIG. 1, a brace 100 may include a combination of a knit portion (e.g., sock), a shank (which may be rigid), an adjustable fit system, and a soft cushioned heel (e.g., a pad). The shank 1 may be a rigid plastic shank at the bottom of the brace 100 that provides support to the plantar tendon from the calcaneus to the metatarsals. The support from the shank 1 helps to unload the plantar tendon, allowing for a faster recovery. The woven or crisscross structure of the shank 1, as seen in the views of FIG. 2 and FIG. 3, allows for rigidity from toe to heel while allowing the sides to bend and conform to each individual foot. The shank 1 may be configured in a horseshoe shape 9 at the portion that wraps around the heel to contain the corpus adiposum (e.g. natural fat pad in the heel). This configuration may serve to provide support and enhance the natural shock absorption of the fat pad and it may keep the shank 1 in the correct location during activity.

In embodiments, the shank 1 may comprise glass-reinforced nylon, a polyether block amide (PEBA) or other thermoelastic polymer, a thermoplastic elastomer, a thermoplastic urethane, an ethylene vinyl acetate, a poly (ethylene vinyl acetate), a carbon fiber or any rigid plastic. In embodiments, the thickness may be between about 1 mm to about 2.5 mm thick. In certain embodiments, the thickness may exceed 2.5 mm. Holes 15 or other openings may be disposed in and distributed throughout the shank 1 and/or sock 2 to allow for comfort and breathability as heat vapor and moisture from sweat are dissipated from the bottom of the foot. In some embodiments, the holes 15 may be about 10 mm².

In embodiments, the configuration of the shank 1 may be a cradle weave 14. The cradle weave 14 may be flatter on the lateral side of the foot to reduce irritation. Referring to FIG. 2, the cradle weave 16 on the medial side may wrap the foot further to increase arch support. The cradle weave 14 structure allows for support and some flexibility to allow the shank 1 to adapt to the contours of different feet, such as low, medium or high arches. In some embodiments, the encroachment of the shank 1 up the sides of the foot may be equivalent on both sides to enable a brace 100 that can be used on either foot.

In embodiments, the shank 1 may be adhered to or otherwise joined or associated with a flat knit or circular knit sock 2 that provides a base structure of support while offering comfort. The sock 2 may be generally contoured to correspond to an ankle and/or foot structure. The sock 2 may provide thin and comfortable containment, keeping the shank 1 in place and providing for attachment of the webbing support system 3 and the lacing system 4. The knit sock 2 may comprise various threads or yarns which may be used to enhance breathability, support and flexibility. The yarns used may be polyester, spandex, thermo plastic urethane, monofilament yarns, carbon fiber, and/or cotton. In some embodiments, odor absorbent or wicking materials may be used. When placed on a foot, the sock 2 may be situated directly behind the metatarsal heads (or the ball of the foot), contain the majority of the foot, and extend to above the malleolus. In some embodiments, the sock 2 may cover the toes.

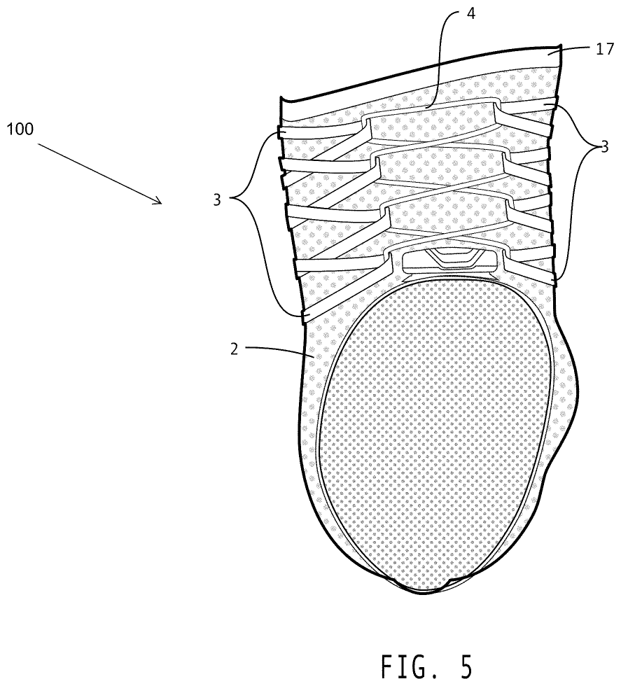

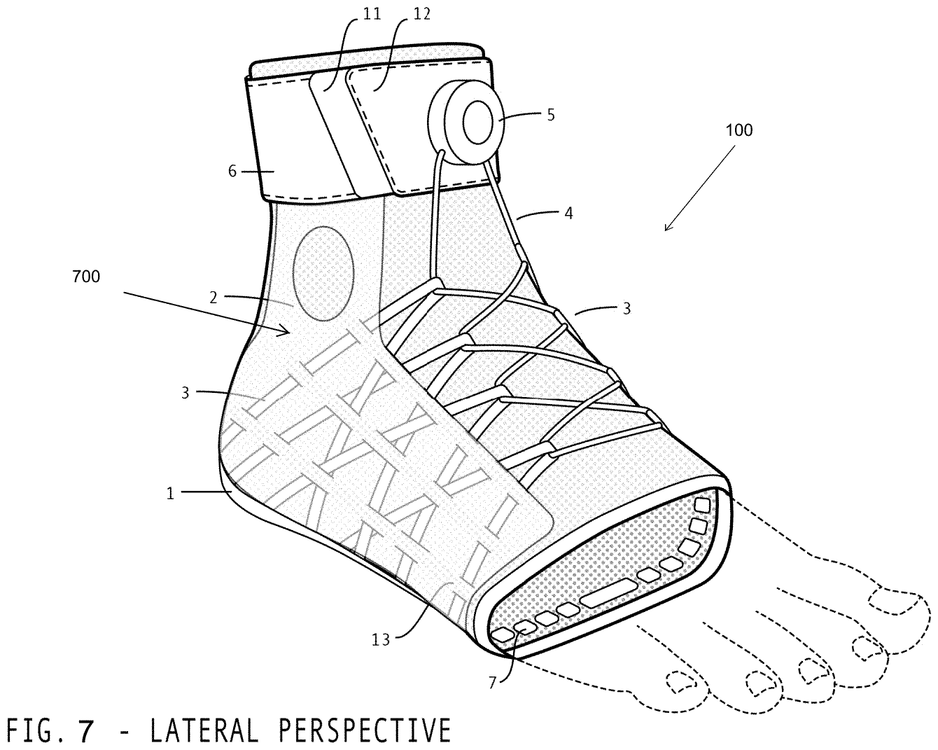

Attached to the sock 2 and the shank 1 may be a support system 3, which may include webbing (referred to herein as a webbing support system) formed from one or more of nylon or elastomeric bands, polyester yarns or thermoplastic bands. While the support system 3 is depicted herein as including webbing, it is to be understood that embodiments of the support system 3 may include features other than webbing (e.g., the support system may be a substantially monolithic and/or solid piece of material). The crisscross or woven structure of the webbing support system 3 may mirror or replicate a foot and/or ankle taping. The loops of the webbing support system 3 may wrap around the lateral side of the foot, the bottom of the foot and, as seen in FIGS. 3 and 5, up the medial side of the foot. In embodiments, there may be openings 13 in the sock 2 or portions on the surface of the sock 2 through which the webbing support system 3 may be weaved, threaded or attached. In other embodiments, and referring to FIG. 7, a TPU film 700 may be welded over the webbing loops to secure them in place.

In embodiments, the webbing support system 3 may comprise a single piece of webbing continuously forming the webbing support system 3 and being attached to the sock 2 and disposed between the shank 1 and the sock 2. In some embodiments, the single piece of continuous webbing may be adhered to the shank 1 and/or sock 2 so that it doesn't move when the webbing support system 3 is tightened.

In other embodiments, the webbing support system 3 may comprise multiple portions of webbing, with the ends of each individual portion of webbing being attached to the shank 1 and/or sock 2. In embodiments, portions of the webbing may overlap with one another. Referring to FIGS. 2 and 3, portions of the webbing support system 3 may pass between the shank 1 and the sock 2, and in some embodiments, may be adhered to the shank 1 and/or the sock 2 at least along a portion of the webbing. In embodiments, the webbing portions may extend from an edge of the foot up the lateral side of the foot, or extend from an arch of the foot up the medial side of the foot, up towards the top medial portion of the foot. In embodiments, webbing portions may extend from the lateral extensor digitorum longus tendon around the peroneus longus retinaculum tendon, under the bottom of the foot to support the plantar tendon and up the arch and medial side of the foot to the extensor hallucis longus tendon. Webbing portions may form a loop at the top medial portion of the foot. In other embodiments, the webbing portions may individually terminate in a loop, have eyelets, or other configuration that allows for attachment to a lacing system.

The webbing support system 3 may be attached to a lacing system 4 having a lace or multiple laces. The laces may include one or more of cotton, polyester or nylon cord. The lace or laces of the lacing system 4 may be threaded through the top of the webbing loops of the webbing support system 3. In some embodiments, the lacing system 4 may be integrated with the sock 2 to work as a single system. Portions of the lacing system 4 may attach to or terminate in a tightening system 5, which may be a ratchet system, barrel lock or adjustable clip. The tightening system 5 may be used to loosen or tighten the fit of the brace 100 by loosening or tightening the laces of the lacing system 4, which in turn relaxes or cinches the webbing support system 3 and completes the loop around the foot. Tightening the webbing support system 3 pulls the webbing loops towards the top of the foot on both sides of the foot.

Placement of the tightening system 5 on the ankle portion of the ankle foot orthosis enables adjustment while the user is wearing footwear over the ankle foot orthosis 100. The configuration of the webbing support system 3, lacing system 4, and ratchet 5 allows for individual adjustment and custom fitting around each person's unique anatomy in a strong and lightweight package.

In embodiments, individual laces of the lacing system 4 may attach to one of a plurality of tightening systems 5 for adjustment of the portion of the webbing support system 3 threaded through or otherwise associated with the lace. In this way, if differential tightening or support is desired across the ankle/foot, the user may be able to adjust each tightening system 5 of the plurality individually to arrive at the desired differential support.

In embodiments, the brace 100 may be secured and tightened without use of a ratchet 5. A lace may be threaded through the webbing support system 3 and manually tightened and secured in similar fashion to a shoelace tightening a shoe.

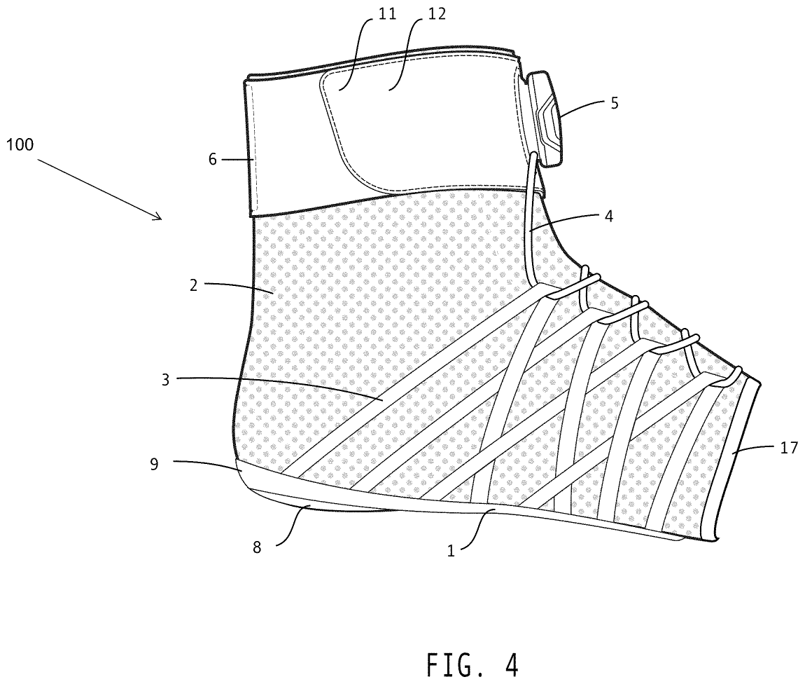

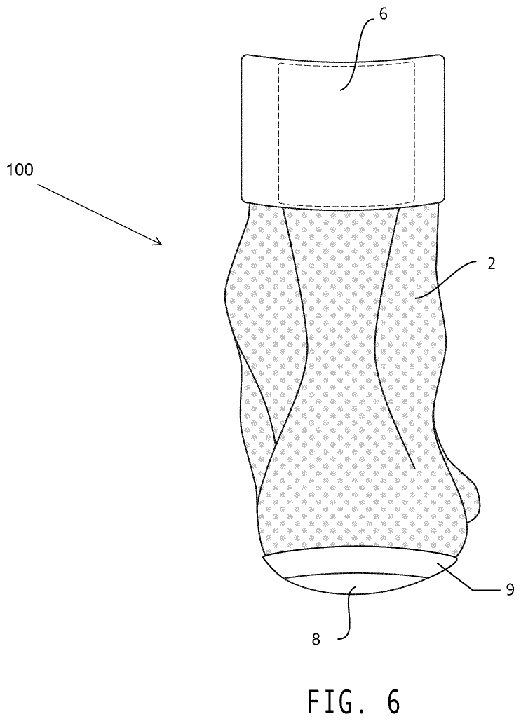

In an embodiment, and referring to FIGS. 1, 3, 4, and 6, an ankle strap 6 provides an anchor point at the top of the brace 100 and provides quick release and tight ankle lock down. The ankle strap 6 may be adjustable to accommodate various ankle dimensions. The adjustment system 11, or closure mechanism, provided by the ankle strap 6 may include laces, loops, a hook and loop fastener, elastic, fasteners, or a combination thereof. For example, a hook and loop fastener-based adjustment system 11 may allow for quick release and ankle lock down. A top strap 12 may also have an attachment system, such as a hook and loop fastener or other system as described herein, for quick release and ankle lock down. In embodiments, the attachment system 11 includes the loops and the top strap 12 includes the hooks of a hook-and-loop fastener. The ankle strap 6 may be made from leather, synthetic leather, suede, synthetic suede, a microfiber, or a combination thereof, or any other suitable material.

Referring to FIG. 1, protrusions 7, such as silicon beads, may be disposed anywhere on the inside of the sock 2 to help reduce slipping and to ensure the brace 100 stays in the correct location during activity. The protrusions 7 may be disposed along an edge of the brace 100. The protrusions 7 may be disposed on the top and bottom of the sock 2.

In embodiments, a soft, closed cellular foam urethane pad 8, such as made from polyurethane, EVA—Ethylene Vinyl Acetate, or gel may be positioned under the calcaneus to cushion the heel on impact. The pad 8 may provide additional lift to the heel taking weight and pressure off of the plantar tendon. In certain embodiments, the pad 8 may be tear-drop shaped so that it transitions from comfort and cushioning directly under the center of the heel, and as the shape tapers, it provides more support and containment at the point of transition between the calcaneus and the plantar tendon. It should be understood that the pad 8 can be any shape. In embodiments, portions of the shank 1 may wrap around the pad 8. In these embodiments, the pad 8 may be attached directly to the sock 2. In other embodiments, the pad 8 may be disposed below or above the shank 1.

Referring to FIGS. 3, 4, and 5, the sock 2 may be finished with a finishing 17, such as a binding tape, a flat knit stitch or a welded edge, to prevent fraying edges. The finishing 17 may also help to locate the protrusions 7 at the edges of the knit structure of the sock 2.

In operation, a user experiencing the need for ankle or foot support may use the brace 100 by inserting their foot into the brace so that it is positioned on a portion of the foot and ankle with the pad 8 below the heel and by securing the brace 100 using the ankle strap 6. Once the brace 100 is placed on the ankle, the user can customize the fit. Tightening the lacing system 4 applies a force to the webbing system 3 to cinch it further around the foot, increasing the support provided by the brace 100, which can be done while conventional socks and shoes are worn over the brace 100. To relax the tension on the webbing system 3, the user loosens the lacing system 4 thus loosening the webbing support system 3.

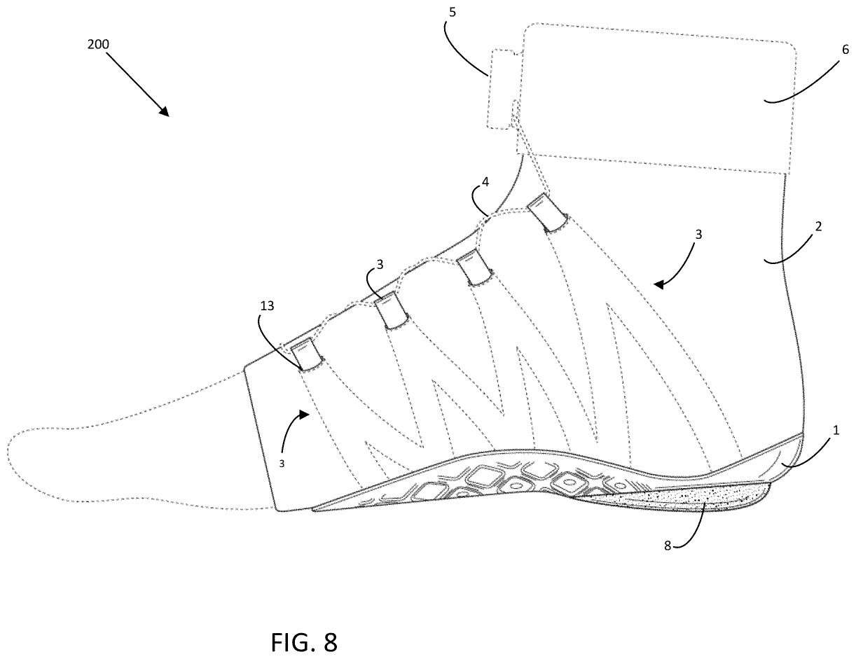

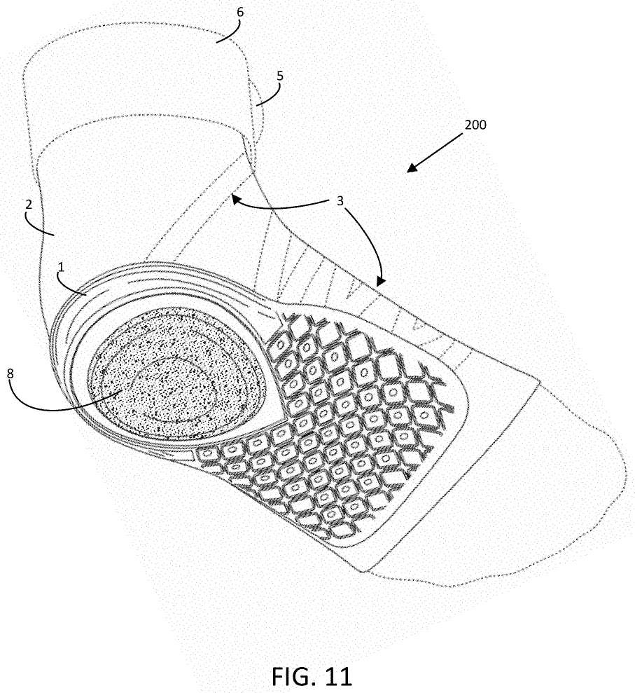

Shown in FIGS. 8-11 is another embodiment of a brace 200 wherein the support system 3 may be integrated into and/or underneath the knit portion 2 (depicted by the dashed portions of the support system 3). As will be understood, covering a large portion of the support system 3 with the knit portion 2 protects the support system 3 and/or mitigates the likelihood that the support system 3 will become snagged on an object, (e.g., door stops, sticks, rocks, sporting equipment, etc.).

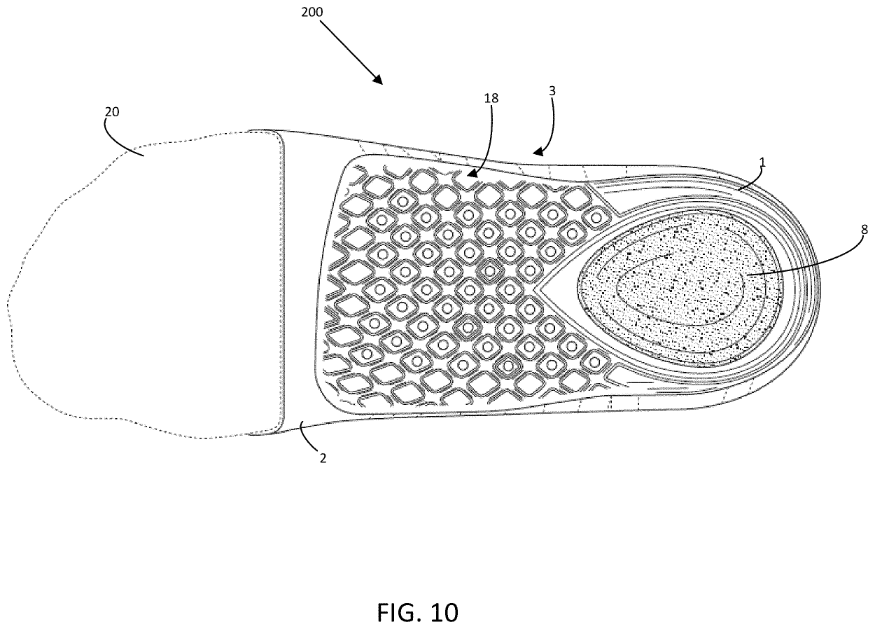

As best seen in FIG. 10, embodiments of the shank 1 may include a tread 18. The tread 18 may be of a pattern the same as and/or similar to the one shown in FIGS. 8, 10, and 11, and/or it may be of a different pattern. Embodiments of the shank 1 having a tread 18 may provide for better traction of the brace 200 on walking surfaces.

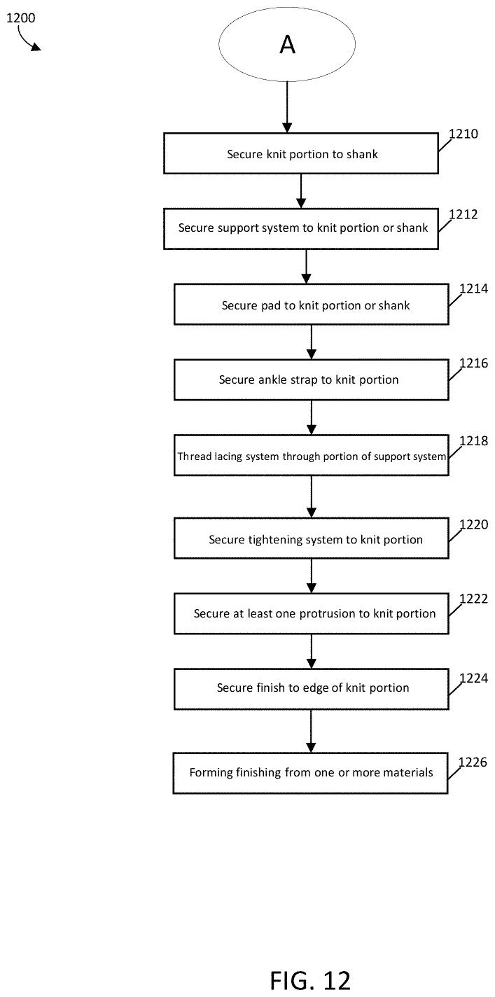

Referring to FIG. 12, a method 1200 for manufacturing a brace (e.g., 100 and/or 200) includes securing 1210 the knit portion 2 to the shank 1 and securing 1212 the support system 3 to the shank 1 and/or the knit portion 2. While FIG. 12 depicts securing 1210 the knit portion 2 to the shank 1 as occurring before securing 1212 the support system 3 to the knit portion 2 and/or the shank 1, it will be understood that securing 1212 of the support system 3 to the knit portion 2 and/or the shank 1 may occur before securing 1210 of the knit portion 2 to the shank 1. Securing 1210 of the knit portion 2 to the shank 1 may include adhering the knit portion 2 to the shank 1 via an adhesive, sewing the knit portion 2 to the shank 1, clamping the knit portion 2 to the shank 1, and/or any other suitable manner of securing the knit portion 2 to the shank 1. Similarly, securing 1212 the support system 3 to the knit portions 2 and/or the shank 1 may also include adhering the support system 3 to the knit portion 2 and/or the shank 1 via an adhesive, sewing the support system 3 to the knit portions 2 and/or the shank 1, clamping the support system 3 to the knit portion 2 and/or the shank 1, and/or any other suitable manner of securing the support system 3 to the knit portion 2 and/or the shank 1.

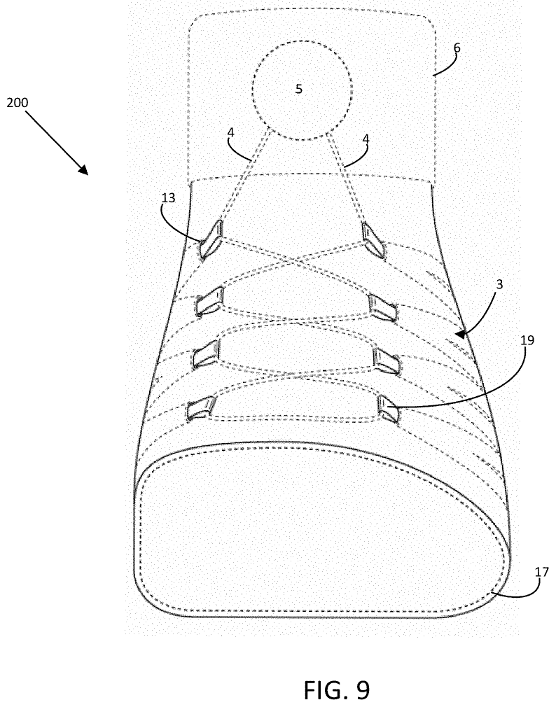

The method 1200 may further include securing 1214 the pad 8 to the knit portion 2 and/or the shank 1. The pad 8 may be secured 1214 via an adhesive, sewed, clamped and/or otherwise secured to the knit portion 2 and/or the shank 1. The method 1200 may further include securing 1216 the ankle strap 6 to the knit portion 2. The ankle strap 6 may be secured 1216 via an adhesive, sewed, clamped and/or otherwise secured to the knit portion 2. The method 1200 may further include threading 1218 the lacing system 4 through a portion (e.g., loops 19 (FIGS. 1 and 9)) at a location corresponding to a top medial portion of a foot 20 (FIGS. 1 and 10). The method 1200 may further include securing 1220 the tightening system 5 to the knit portion 2. The method 1200 may further include securing 1222 at least one protrusion 7 to the knit portion 2. The method 1200 may further include securing 1224 a finishing 17 to an edge of the knit portion 2; and/or forming 1226 the finishing 17 from one or more materials (e.g., out of the knit portion 2).

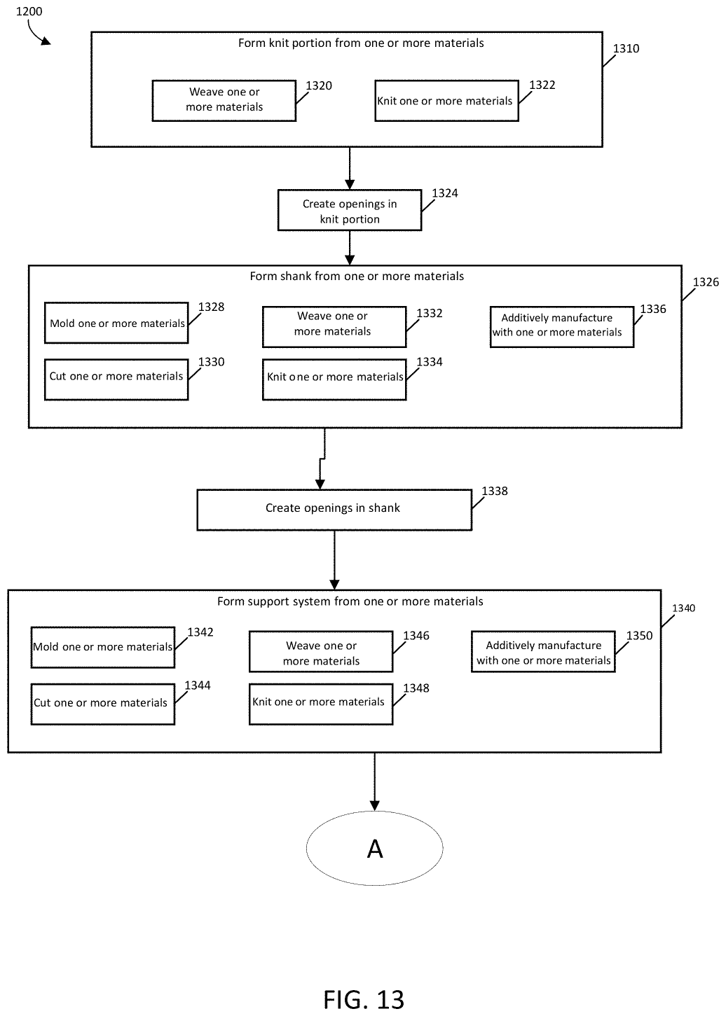

As shown in FIG. 13, embodiments of the method 1200 may include forming 1310 the knit portion 2 from one or more materials. The knit portion 2 may be formed 1310 via weaving 1320 and/or knitting 1322 the one or more materials. Holes 15 may be created 1324 in the knit portion 2 (e.g., via cutting, punching, burning, ablating, etc.).

The method 1200 may include forming 1326 the shank 1 from one or more materials. Forming 1326 the shank 1 may include molding 1328, cutting, 1330, weaving 1332, knitting 1334, and/or additively manufacturing 1336 (with) the one or more materials. Holes 15 may be created 1338 in the shank 1 (e.g., via cutting, punching, burning, ablating, etc.).

The method 1200 may include forming 1340 the support system 3 from one or more materials. Forming 1340 the support system 3 may include molding 1342, cutting 1344, weaving 1346, knitting 1348, and/or additively manufacturing 1350 (with) the one or more materials.

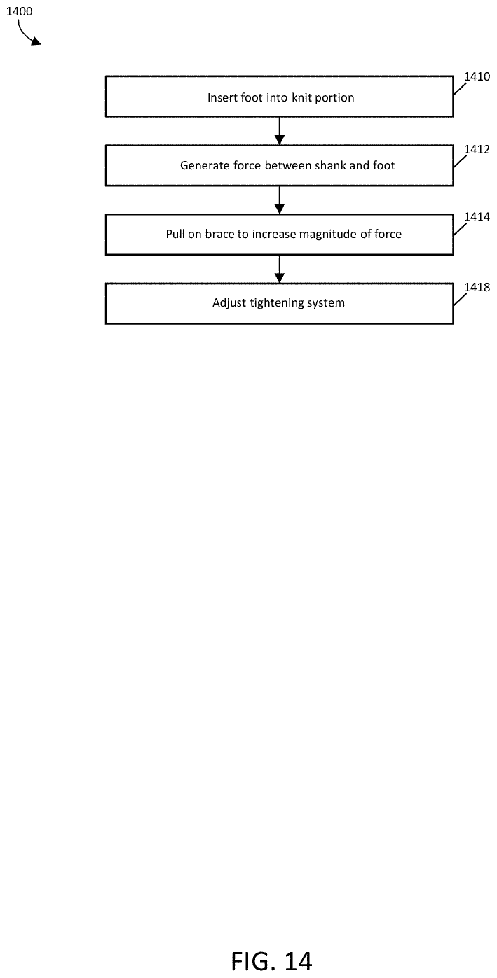

A method 1400 for bracing a foot (e.g., using the brace (100 or 200)) is shown in FIG. 14. The method 1400 includes inserting 1410 a foot into the knit portion 2 such that at least a portion of the foot is covered by the shank 1. The method 1400 further includes generating 1412, via the support system 3, a force between the shank 1 and the foot. The method 1400 may include pulling 1414 on the brace (100 or 200) to increase a magnitude of the force. For example, a wearer of the brace (100 or 200) may pull on the brace (100 or 200) so that the brace (100 or 200) pulls on a tendon or other foot structure. As will be appreciated, when pulled so as to generate the force, the brace (100 or 200) serves as an adjustable counter-orthotic. It is also to be understood that a pushing action, or any other action that increases the magnitude of the force between the shank 1 and the foot, may be used in place of and/or in addition to the act of pulling 1414 on the brace. In embodiments, the method 1400 may include adjusting 1418 the tightening system 5.

Certain further aspects of the example brace and methods are described following, any one or more of which may be present in certain embodiments.

An example brace includes a knit portion, a shank, and a support system. The knit portion is contoured for a foot. The shank is contoured for a bottom of the foot so as to cover at least a portion of an arch of the foot. The support system extends along at least part of the knit portion and is structured to generate a force between the shank and the foot. In certain embodiments, the support system is structured to extend from a lateral extensor digitorum longus tendon of the foot around a peroneus longus retinaculum tendon of the foot, under a bottom of the foot, and up the arch and a medial side of the foot to an extensor hallucis longus tendon of the foot. In certain embodiments, the brace further includes a pad disposed on at least one of the knit portion or the shank at a location corresponding to a heel of the foot. In certain embodiments, the pad includes a closed cellular foam urethane. In certain embodiments, the pad has a tear-drop shape. In certain embodiments, the tear-drop shape is tapered. In certain embodiments, the brace further includes an ankle strap structured to secure the knit portion to an ankle associated with the foot. In certain embodiments, the brace further includes a lacing system threaded through a portion of the support system at a location corresponding to a top medial portion of the foot. In certain embodiments, the brace further includes a tightening system structured to apply a force on the lacing system to cinch the support system against the foot. In certain embodiments, the tightening system includes at least one of: a rachet system; a barrel lock; or an adjustable clip. In certain embodiments, the brace further includes an ankle strap structured to secure the knit portion to an ankle associated with the foot. In such embodiments, the tightening system is disposed on the ankle strap. In certain embodiments, the tightening system is disposed on the knit portion. In certain embodiments, the support system includes webbing. In certain embodiments, the webbing includes a plurality of individual webbing portions each configured to be threaded by the lacing system. In certain embodiments, the plurality of individual webbing portions form a plurality of loops at a location corresponding to a top medial portion of the foot, and the brace further includes a thermoplastic polyurethane film welded over the plurality of loops. In certain embodiments, the webbing weaves through one or more openings in the knit portions. In certain embodiments, the webbing is a single continuous piece of webbing that forms a majority of the support system. In certain embodiments, portions of the lacing system attach to the tightening system. In certain embodiments, the portions of the lacing system terminate at the tightening system. In certain embodiments, individual laces of the lacing system attach to one of a plurality of tightening systems for adjustment of a portion of the support system. In certain embodiments, the individual laces are threaded through the portion of the support system. In certain embodiments, the support system is integrated into the knit portion. In certain embodiments, the support system is disposed between the shank and the knit portion. In certain embodiments, the shank is structured to be flat on a lateral side of the foot. In certain embodiments, the shank is structured to encroach medially on the foot to wrap around the arch of the foot. In certain embodiments, the shank includes a woven structure. In certain embodiments, the woven structure is a cradle weave. In certain embodiments, the shank has a horseshoe-shaped portion structured to wrap around a heel of the foot to support a corpus adiposum of the foot. In certain embodiments, the shank has a thickness of about 1 mm to about 2.4 mm. in certain embodiments, the shank has a thickness greater than or equal to about 2.5 mm. In certain embodiments, the brace further includes at least one protrusion disposed on the knit portion and structured to restrict movement of the foot with respect to the knit portion. In certain embodiments, the at least one protrusion includes a silicon bead. In certain embodiments, the brace further includes a finishing on an edge of the knit portion. In certain embodiments, the finishing includes one or more of: a binding shape; a flat shape; or a wedge edge. In certain embodiments, the knit portion includes a plurality of openings structured to provide breathability for the foot. In certain embodiments, the shank includes a plurality of openings structured to provide breathability for the foot. In certain embodiments, the knit portion includes at least one of: a polyester; a spandex; a thermoplastic urethane; a monofilament yarn; or a cotton. In certain embodiments, the support system includes of at least one of: a nylon band; an elastomeric band; a polyester yarn; or a thermoplastic band. In certain embodiments, the shank includes at least one of: a glass-reinforced nylon; a thermoelastic polymer; a thermoplastic elastomer; a thermoplastic urethane; an ethylene vinyl acetate; a poly ethylene vinyl acetate; a carbon fiber; or a rigid plastic. In certain embodiments, the brace further includes an ankle strap structured to secure the knit portion to an ankle associated with the foot. In such embodiments, the ankle strap includes at least one of: a leather; a synthetic leather; a suede; a synthetic suede; a microfiber; or a combination thereof.

An example method of manufacturing a brace includes securing a knit portion to a shank, the knit portion contoured for a foot, the shank contoured for a bottom of the foot so as to cover at least a portion of an arch of the foot; and securing a support system to at least one of the knit portion or the shank. The support system extends along at least a part of the knit portion and is structured to generate a force between the shank and the foot. In certain embodiments, the support system is structured to extend from a lateral extensor digitorum longus tendon of the foot around a peroneus longus retinaculum tendon of the foot, under a bottom of the foot, and up the arch and a medial side of the foot to an extensor hallucis longus tendon of the foot. In certain embodiments, the method further includes securing a pad to at least one of the knit portion or the shank at a location corresponding to a heel of the foot. In certain embodiments, the method further includes securing an ankle strap to the knit portion, wherein the ankle strap is structured to secure the knit portion to an ankle associate with the foot. In certain embodiments, the method further includes threading a lacing system through a portion of the support system at a location corresponding to a top medial portion of the foot. In certain embodiments, the method further includes securing a tightening system to the knit portion, the tightening system structured to apply a force on the lacing system to cinch the support system against the foot. In certain embodiments, the method further includes securing at least one protrusion to the knit portion. In such embodiments, the knit portion is structured to restrict movement of the foot with respect to the knit portion. In certain embodiments, the method further includes securing a finishing to an edge of the knit portion. In certain embodiments, the method further includes forming the knit portion from one or more materials. In certain embodiments, forming the knit portion includes at least one of: weaving the one or more materials to form the knit portion; or knitting the one or more materials to form the knit portion. In certain embodiments, the method further includes creating a plurality of openings in the knit portion, the plurality of openings structured to provide breathability for the foot. In certain embodiments, the method further includes forming the shank from one or more materials. In certain embodiments, forming the shank includes at least one of: molding the one or more materials; cutting the one or more materials; weaving the one or more materials; knitting the one or more materials; or additively manufacturing with the one or more materials. In certain embodiments, the method further includes creating a plurality of openings in the shank, the plurality of openings structured to provide breathability for the foot. In certain embodiments, the method further incudes forming the support system from one or more materials. In certain embodiments, forming the support system includes: molding the one or more materials; cutting the one or more materials; weaving the one or more materials; knitting the one or more materials; or additively manufacturing with the one or more materials.

An example method of bracing a foot includes inserting the foot into a knit portion of a brace such that at least a portion of an arch of the foot is covered by a shank of the brace; and generating, via a support system of the brace, a force between the shank and the foot, the support system extending along at least a part of the knit portion. In certain embodiments, the support system is structured to extend from a lateral extensor digitorum longus tendon of the foot around a peroneus longus retinaculum tendon of the foot, under a bottom of the foot, and up the arch and a medial side of the foot to an extensor hallucis longus tendon of the foot. In certain embodiments, the method further includes pulling on the brace to increase a magnitude of the force. In certain embodiments, generating, via a support system of the brace, a force between the shank and the foot, the support system extending along at least part of the knit portion includes adjusting a tightening system of the brace. In certain embodiments, the tightening system cinches the support system against the foot via a lacing system of the brace.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

The use of the terms “a” and “an” and “the” and similar referents in the context of describing the disclosure (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure, and does not pose a limitation on the scope of the disclosure unless otherwise claimed. No language in the specification should be construed as indicating any non-claimed element as essential to the practice of the disclosure.

While the foregoing written description enables one skilled in the art to make and use what is considered presently to be the best mode thereof, those skilled in the art will understand and appreciate the existence of variations, combinations, and equivalents of the specific embodiment, method, and examples herein. The disclosure should therefore not be limited by the above described embodiment, method, and examples, but by all embodiments and methods within the scope and spirit of the disclosure.

Any element in a claim that does not explicitly state “means for” performing a specified function, or “step for” performing a specified function, is not to be interpreted as a “means” or “step” clause as specified in 35 U.S.C. § 112(f). In particular, any use of “step of” in the claims is not intended to invoke the provision of 35 U.S.C. § 112(f).

Persons skilled in the art may appreciate that numerous design configurations may be possible to enjoy the functional benefits of the inventive systems. Thus, given the wide variety of configurations and arrangements of embodiments of the present invention, the scope of the invention is reflected by the breadth of the claims below rather than narrowed by the embodiments described above.

1. A brace, comprising:
a knit portion contoured for a foot;
a shank contoured for a bottom of the foot so as to cover at least a portion of an arch of the foot; and
a support system extending along at least part of the knit portion and structured to generate a force between the shank and the foot.

2. The brace of claim 1, wherein the support system is structured to extend from a lateral extensor digitorum longus tendon of the foot around a peroneus longus retinaculum tendon of the foot, under a bottom of the foot, and up the arch and a medial side of the foot to an extensor hallucis longus tendon of the foot.

3. The brace of claim 1 further comprising:
a pad disposed on at least one of the knit portion or the shank at a location corresponding to a heel of the foot.

4. The brace of claim 3, wherein the pad comprises a closed cellular foam urethane.

5. The brace of claim 3, wherein the pad has a tear-drop shape.

6. The brace of claim 5, wherein the tear-drop shape is tapered.

7. The brace of claim 1 further comprising:
an ankle strap structured to secure the knit portion to an ankle associated with the foot.

8. The brace of claim 1 further comprising:
a lacing system threaded through a portion of the support system at a location corresponding to a top medial portion of the foot.

9. The brace of claim 8 further comprising:
a tightening system structured to apply a force on the lacing system to cinch the support system against the foot.

10. The brace of claim 9, wherein the tightening system comprises at least one of:
a rachet system;
a barrel lock; or
an adjustable clip.

11. The brace of claim 9 further comprising:
an ankle strap structured to secure the knit portion to an ankle associated with the foot;
wherein the tightening system is disposed on the ankle strap.

12. The brace of claim 9, wherein the tightening system is disposed on the knit portion.

13. The brace of claim 9, wherein the support system comprises webbing.

14. The brace of claim 13, wherein the webbing compromises a plurality of individual webbing portions each configured to be threaded by the lacing system.

15. The brace of claim 14, wherein:
the plurality of individual webbing portions form a plurality of loops at a location corresponding to a top medial portion of the foot; and
the brace further comprises:
a thermoplastic polyurethane film welded over the plurality of loops.

16. The brace of claim 13, wherein the webbing weaves through one or more openings in the knit portions.

17. The brace of claim 13, wherein the webbing is a single continuous piece of webbing that forms a majority of the support system.

18. The brace of claim 9, wherein portions of the lacing system attach to the tightening system.

19. The brace of claim 18, wherein the portions of the lacing system terminate at the tightening system.

20. The brace of claim 8, wherein individual laces of the lacing system attach to one of a plurality of tightening systems for adjustment of a portion of the support system.

21.-61. (canceled)
